(19) United States
(12) Patent Application Publication
(10) Pub. No.: US 2021/0335448 A1
(43) Pub. Date: Oct. 28, 2021

(54) METHODS OF IDENTIFYING TREATMENTS USING DIFFERENTIALLY EXPRESSED GENES

(71) Applicant: Advaita Corporation, Ann Arbor, MI (US)

(72) Inventor: Sorin DRAGHICI, Ann Arbor, MI (US)

(73) Assignee: Advaita Corporation, Ann Arbor, MI (US)

(21) Appl. No.: 17/242,145

(22) Filed: Apr. 27, 2021

Related U.S. Application Data
(60) Provisional application No. 63/016,078, filed on Apr. 27, 2020.

(51) Int. Cl. G16B 5/20; G16B 25/10; G16B 40/30; A61K 31/573; A61K 31/196

(52) U.S. Cl. CPC G16B 5/20; G16B 25/10; A61K 31/196; A61K 31/573; G16B 40/30

(57) ABSTRACT

The present disclosure provides a method for identifying targeted treatments for treatment of a phenotype in a human or non-human subject. The method may include providing data relating to a plurality of affected biomolecules to one or more processors; the one or more processors, using a first probability, to identifying one or more biological pathways impacted be the affected biomolecules; the one or more processors identifying one or more biological processes affected by the phenotype; the one or more processors, identifying one or more upstream regulators; and the one or more processors, using a second probability, determining at least one targeted treatment configured to reverse expressions of at least two of the affected biomolecules on the one or more biological pathways impacted by the phenotype.

### CROSS-REFERENCE TO RELATED APPLICATION

This application claims the benefit of U.S. Provisional Patent Application No. 63/016,078, filed on Apr. 27, 2020. The entire disclosure of the above application is incorporated herein by reference.

### FIELD

The present disclosure relates methods and systems for identifying treatments using differentially expressed genes, differentially expressed proteins, and/or similar omics data (such as, methylation, microRNA, metabolomics, and the like), and also, methods for treating disorders in a subject applying treatments identified using such identification methods.

### BACKGROUND

This section provides background information related to the present disclosure which is not necessarily prior art.

The emergence of some highly pathogenic coronaviruses has been observed in the twenty-first century. Coronaviruses are a diverse group of single-stranded, positive-sense RNA viruses infecting a wide range of vertebrate hosts. These viruses generally cause mild upper respiratory tract illnesses in humans, such as the common cold. However, three highly pathogenic human coronaviruses have emerged in the past two decades: SARS-CoV-1, which caused severe acute respiratory syndrome (SARS) and infected approximately 8,000 people worldwide with a case-fatality rate of 9.36% in 2002-2003; MERS-CoV, which caused the Middle East respiratory syndrome-related coronavirus (MERS) and infected approximately 2,500 people with a case-fatality rate of 30%; and now SARS-CoV-2, which causes the Coronavirus Disease-2019 (commonly referred to as COVID-19), whose global mortality rate remains to be determined. Infection with these highly pathogenic coronaviruses can result in acute lung injury (“ALI”) and acute respiratory distress syndrome (“ARDS”), often leading to a significant reduction of lung function and even death.

The current pandemic of COVID-19 represents an acute and rapidly developing global health crisis. The growth of the number of cases has skyrocketed globally. This growth has been and, as of this date, continues to be exponential in all but a few countries. The global economy has been brought to an almost complete halt due to the social distancing and other virus mitigation measures taken everywhere.

The current potential COVID-19 treatments being tested include anti-virals, anti-malarial drugs and other compounds that may prevent viral replication. Anti-virals may be effective to limit the length of infection in most people, as well as reduce the transmission between individuals. However, for the patients who develop severe and/or critical disease, and who are destined for intensive care, anti-virals alone may not help. These patients could already be in a “system hyperinflammation” stage and facing pneumonia, acute respiratory distress syndrome (“ARDS”), septic shock, and multi-organ failure; conditions that are not caused by the virus directly, but rather by the host response to the virus. Accordingly, methods of identifying appropriate and effective treatments, and methods of treating disorders using the same, are desirable.

### SUMMARY

This section provides a general summary of the disclosure, and is not a comprehensive disclosure of its full scope or all of its features.

The present disclosure is related to methods and systems for identifying appropriate and effective treatments using differentially expressed genes differentially expressed proteins, and/or similar omics data (such as, methylation, microRNA, metabolomics, and the like), and also, methods and systems for treating disorders (e.g., coronaviruses and similar viruses or infections agents, including such infections agents associated with aberrant immune responses) in a subject adopting treatments identified using such identification methods

In various aspects, the present disclosure provides a method for identifying targeted treatments for treatment of a phenotype in a human or non-human subject. The method may include providing data relating to a plurality of affected biomolecules to one or more processors; the one or more processors, using a first probability, to identifying one or more biological pathways impacted be the affected biomolecules; the one or more processors, identifying one or more upstream regulators in each of the one or more biological pathways; and the one or more processors, using a second probability, determining at least one targeted treatment configured to reverse expressions of at least one of the affected biomolecules and the one or more biological pathways.

In one aspect, the data relating to the plurality of affected biomolecules may include p-values for each of the affected biomolecules, and the method may further include the one or more processors, obtaining a first data collection relating to a first plurality of biomolecules in affected subjects and a second data collection relating to the first plurality of biomolecules in unaffected subjects; and the one or more processors, using the first data collection and the second state collection, determining p-values for each of the affected biomolecules.

In one aspect, the method may further include preparing the data relating to the plurality of affected biomolecules, where the data is prepared by comparing first expression levels of a plurality of expressed genes in affected subjects and second expression levels of the plurality of expressed genes in unaffected subjects.

In one aspect, the data relating to the plurality of affected biomolecules may define a plurality of differentially expressed biomolecules.

In one aspect, the first probability includes a first probability value (pORA) that is a probability of observing a number of the differentially expressed biomolecules in a respective biological pathway of the one or more biological pathways just by chance; and a second probability value (pACC) that is the probability of observing a total perturbation accumulation as measured in each of the respective biological pathways of the one or more biological pathways just by chance.

In one aspect, the method further includes combining the first probability value (pORA) and the second probability value (pACC) so as to determine a unique pathway-specific uncorrected p-value for each of the one or more biological pathways.

In one aspect, the targeted treatment may have a p-value that is less than a predetermined threshold of significance and reverses expression of more than two of the respective differentially expressed biomolecules.

In one aspect, the method may further include, prior to the identifying of the targeted treatment, the one or more processors, using a putative mechanism inference approach, determining mechanisms involved within the respective biological pathway of the one or more biological pathways and linking one or more of the plurality of differentially expressed genes.

In one aspect, the putative mechanism inference may be further based on at least one of measured fold changes in the respective biomolecules of the plurality of biomolecules or the respective biological pathways of the one or more biological pathways and known protein-protein interactions (“PPIs”) for the respective biological pathways of the one or more biological pathways.

In one aspect, the method may further include treating an infection associated with a corona virus in a human or non-human subject. The treating may include administering to the subject a therapeutically effective amount of a composition including a compound selected from the group consisting of: prednisolone, dexamethasone, diclofenac, myochrsine, esters thereof, derivatives thereof, salts thereof, and combinations thereof.

In one aspect, the composition may be a first composition and the compound may be a first compound, and the treating may further include administering to the subject a therapeutically effective amount of a second composition comprising a second compound, where the second compound is selected from the group consisting of: methylprednisolone, tofacitinib, dexamethasone prednisolone, diclofenac, myochrysine, esters thereof, derivatives thereof, salts thereof, and combinations thereof. The second composition is different from the first composition and the administering to the subject the therapeutically effective amount of a second composition may be performed simultaneously with or separately from the administering of the first composition.

In one aspect, the treating may further include coadministering an adjunct therapeutic agent to the subject. The coadministering may be performed simultaneously with or separately from the administering the composition.

In one aspect, the compound may include prednisolone, an ester thereof, a derivative thereof, or a salt thereof, and the administering of the composition may include administering to the subject a dosage of greater than or equal to about 0.1 mg to less than or equal to about 300 mg per day.

In one aspect, the compound may include diclofenac, an ester thereof, a derivative thereof, or a salt thereof, and administering of the composition may include administering to the subject a dosage of greater than or equal to about 1 mg to less than or equal to about 250 mg per day.

In one aspect, the compound comprises myochrysine, an ester thereof, a derivative thereof, or a salt thereof, and administering of the composition may include administering to the subject a dosage of greater than or equal to about 1 mg to less than or equal to about 1 g per week.

In various aspects, the present disclosure provides a method for identifying targeted drug treatments for treatment of a phenotype in a human or non-human subject. The method may include one or more processors identifying one or more biomolecules affected by the phenotype by isolating a plurality of biomolecules differentially expressed between the phenotype and a control; the one or more processors, using one or more first probabilities, identifying one or more biological pathways associated with each of the differentially expressed biomolecules; the one or more processor, identifying one or more upstream regulators; the one or more processors, using a putative mechanism inference approach, determining mechanisms involved with the respective biological pathway of the one or more biological pathways or linking one or more of the plurality of differentially expressed biomolecules; and the one or more processors, using one or more second probabilities, determining at least one targeted drug treatment configured to reverse expressions of at least two of the biomolecules that are differentially expressed in the given phenotype.

In one aspect, using the one or more second probabilities may include determining a p-value indicative of the chance likelihood of reversing the expressions of the biomolecules in the one or more biological pathways, and the targeted drug treatment may have a p-value that is less than a predetermined threshold of significance.

In one aspect, the method may further include treating an infection associated with a corona virus in a human or non-human subject. The treating may include administering to the subject a therapeutically effective amount of a composition including a compound selected from the group consisting of: methylprednisolone, tofacitinib, dexamethasone prednisolone, diclofenac, myochrysine, esters thereof, derivatives thereof, salts thereof, and combinations thereof.

In various aspects, the present disclosure provides a method for identifying targeted drug treatments for treatment of a phenotype in a subject. The method may include one or more processors identifying one or more biomolecules affected by the phenotype by isolating a plurality of differentially expressed genes associated with each of the one or more biomolecules; the one or more processors determining a first probability value (pORA) that is a probability of observing one or more of the plurality of differentially expressed genes in a biological pathway; the one or more processors determining a second probability value (pACC) that reflects a total accumulation of the plurality of differentially expressed genes as measured in the biological pathway; the one or more processors combining the first probability value (pORA) and the second probability value (pACC) so as to determine a unique pathway-specific uncorrected p-value for the biological pathway; the one or more processors identifying one or more upstream regulators, where the one or more upstream regulators are upstream of a respective biomolecule; the one or more processors, using a putative mechanism inference approach, determining mechanisms involved with the biological pathway or linking one or more of the plurality of differentially expressed genes; and the one or more processors, using one or more second probabilities, determining at least one targeted drug treatment configured to reverse expressions of at least two target biomolecules found to be differentially expressed in the give phenotype.

In one aspect, the one or more processors may include a display, and the method may further include displaying at least one of (i) the one or more biomolecules, (ii) the plurality of differentially expressed genes associated with each of the phenotype, (iii) the first probability value (pORA), (iv) the second probability value (pACC), (v) the unique pathway-specific uncorrected p-value for the biological pathway, (vi) the one or more upstream regulators, (vii) the mechanisms involved with the biological pathway or linking one or more of the plurality of differentially expressed genes, (viii) the targeted drug treatment, and (ix) processes thereof.

In one aspect, the method may further include treating an infection associated with a corona virus in a human or non-human subject. The treating may include administering to the subject a therapeutically effective amount of a composition including a compound selected from the group consisting of: methylprednisolone, tofacitinib, dexamethasone prednisolone, diclofenac, myochrysine, esters thereof, derivatives thereof, salts thereof, and combinations thereof.

In various aspects, the present disclosure provides a software program, stored in non-transient computer memory, including a module identifying targeted drug treatments for treatment of a phenotype in a subject. The module may include a first set of instructions automatically determining biomolecules affected by the phenotype by isolating differentially expressed biomolecules associated with each of the phenotype; a second set of instructions automatically determining a first probability value that is a probability of observing the number of differentially expressed biomolecules in a biological pathway just by chance; a third set of instructions automatically determining a second probability value associated with a total accumulation of the differentially expressed genes as measured in the biological pathway; a fourth set of instructions automatically combining the first probability value and the second probability value so as to calculate a unique pathway-specific uncorrected value for the biological pathway; a fifth set of instructions identifying upstream regulators; a sixth set of instructions, using a putative mechanism inference, to determine mechanisms involved with the plurality of differentially expressed biomolecules; a seventh set of instructions determining at least one targeted drug treatment configured to reverse expressions of at least two biomolecules found to be differentially expressed in the phenotype; and an eighth set of instructions displaying the targeted drug treatment determination on an output.

Further areas of applicability will become apparent from the description provided herein. The description and specific examples in this summary are intended for purposes of illustration only and are not intended to limit the scope of the present disclosure.

### BRIEF DESCRIPTION OF THE DRAWINGS

The drawings described herein are for illustrative purposes only of selected embodiments and not all possible implementations, and are not intended to limit the scope of the present disclosure.

FIG. 1 is a flowchart of an example method for identifying appropriate and effective treatments for an infection (e.g., SARS-COV-2).

FIGS. 2A and 2B illustrate differentially expressed genes on a chemokine signaling pathway, where FIG. 2A serves as a ledger for FIG. 2B.

FIG. 3A is a schematic illustrating the effects of a upstream regulator (u) upon two downstream genes donated by circles, where the circle with a plus sign denotes a differentially expressed gene with a positive fold change (i.e., FC>0), the circle with the minus sign denotes a differentially expressed gene with a negative fold change (i.e., FC<0), the arrow with the plus sign denotes an activation effect of the upstream regulator (u) upon the downstream gene, and the arrow with the negative sign denotes an inhibition effect of the upstream regulator (u) upon the downstream gene.

FIG. 3B is a schematic detailed example notations, for example, “MT” represents the set of all measured targets, “DTI” represents the set of differentially expressed targets consistent with an inhibition hypothesis, “MT(u)” represents the set of measured targets of the upstream regulator (u), “DT(u)” represents the set of differentially expressed targets of the upstream regulator (u), and “DTI(u)” represents differentially expressed targets of the upstream regulator (u) consistent with the hypothesis that the upstream regulator (u) is inhibited.

FIG. 4 illustrates an example of a biological processes identified as being significantly perturbed in example infected cells as compared to control or healthy cells.

FIG. 5 illustrates an example of biological pathways in the example infected cells as compared to the control or healthy cells.

FIGS. 6A and 6B illustrate the putative mechanism through which methylprednisolone acts on the identified differentially expressed genes, and also, how those differentially expressed genes influence the identified biological processes, where FIG. 6A serves as a ledger for FIG. 6B.

Corresponding reference numerals indicate corresponding parts throughout the several views of the drawings.

### DETAILED DESCRIPTION

Example embodiments are provided so that this disclosure will be thorough, and will fully convey the scope to those who are skilled in the art. Numerous specific details are set forth such as examples of specific compositions, components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, elements, compositions, steps, integers, operations, and/or components, but do not preclude the presence or addition of one or more other features, integers, steps, operations, elements, components, and/or groups thereof. Although the open-ended term “comprising,” is to be understood as a non-restrictive term used to describe and claim various embodiments set forth herein, in certain aspects, the term may alternatively be understood to instead be a more limiting and restrictive term, such as “consisting of” or “consisting essentially of” Thus, for any given embodiment reciting compositions, materials, components, elements, features, integers, operations, and/or process steps, the present disclosure also specifically includes embodiments consisting of, or consisting essentially of, such recited compositions, materials, components, elements, features, integers, operations, and/or process steps. In the case of “consisting of,” the alternative embodiment excludes any additional compositions, materials, components, elements, features, integers, operations, and/or process steps, while in the case of “consisting essentially of,” any additional compositions, materials, components, elements, features, integers, operations, and/or process steps that materially affect the basic and novel characteristics are excluded from such an embodiment, but any compositions, materials, components, elements, features, integers, operations, and/or process steps that do not materially affect the basic and novel characteristics can be included in the embodiment.

Throughout this disclosure, the numerical values represent approximate measures or limits to ranges to encompass minor deviations from the given values and embodiments having about the value mentioned as well as those having exactly the value mentioned. Other than in the working examples provided at the end of the detailed description, all numerical values of parameters (e.g., of quantities or conditions) in this specification, including the appended claims, are to be understood as being modified in all instances by the term “about” whether or not “about” actually appears before the numerical value. “About” indicates that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters. For example, “about” may comprise a variation of less than or equal to 5%, optionally less than or equal to 4%, optionally less than or equal to 3%, optionally less than or equal to 2%, optionally less than or equal to 1%, optionally less than or equal to 0.5%, and in certain aspects, optionally less than or equal to 0.1%.

In addition, disclosure of ranges includes disclosure of all values and further divided ranges within the entire range, including endpoints and sub-ranges given for the ranges.

Example embodiments will now be described more fully with reference to the accompanying drawings.

The present disclosure provides methods for identifying appropriate and effective treatments for various phenotypes, including diseases and disorders, such as severe acute respiratory syndrome (“SARS”) (e.g., severe acute respirator syndrome coronavirus 2 (SARS-CoV-2), corona virus disease 2019 (commonly refer to as. COVID-19), pneumonia, acute respiratory distress syndrome (“ARDS”), septic shock, multi-organ failure, and combinations thereof as non-limiting examples. Methods for identifying appropriate and effective treatments generally include differentiating specific cellular responses (i.e., understanding the immunological response) and selecting treatments specific to the identified cellular response(s) (i.e., selecting treatment that mitigate or alleviate at least some of the devastating over-reaction of the host's immune system (e.g., cytokine storm)). For example, transcriptome analysis of infected tissues and cell samples may be used to understand the main mediators of the infection (for example, the inflammatory process), and once the affected pathways are characterized, specific drugs can be identified that would mitigate or alleviate some of the devastating over-reaction of the host's immune system.

Such methods for identifying appropriate and effective treatments work to identify target specific immune modifiers, which are often disregarded because it is counter-intuitive to intentionally diminish the immune response in a patient whose immune system is actively fighting a virus. Indeed modulating the immune response is likely unnecessary and counter-productive for patients whose immune system is doing a good job at resolving the infection. However, in various patients whose inflammatory response has become dysregulated, immune modifiers may be lifesaving. If a patient has developed severe respiratory symptoms and is hypoxic, the host response that leads to acute respiratory distress syndrome (“ARDS”), sepsis, and organ failure has already been initiated. At this point, the focus should shift to supporting the patients systems and preventing collapse triggered by hyper-inflammation. The present disclosure provides methods for identifying when a patient is exhibiting a normal immune response (where should be supported), as compared to situations in which the patient is in a hyperinflammation stage (where the immune system must be controlled), and the best potential therapeutic approaches for controlling the immune system.

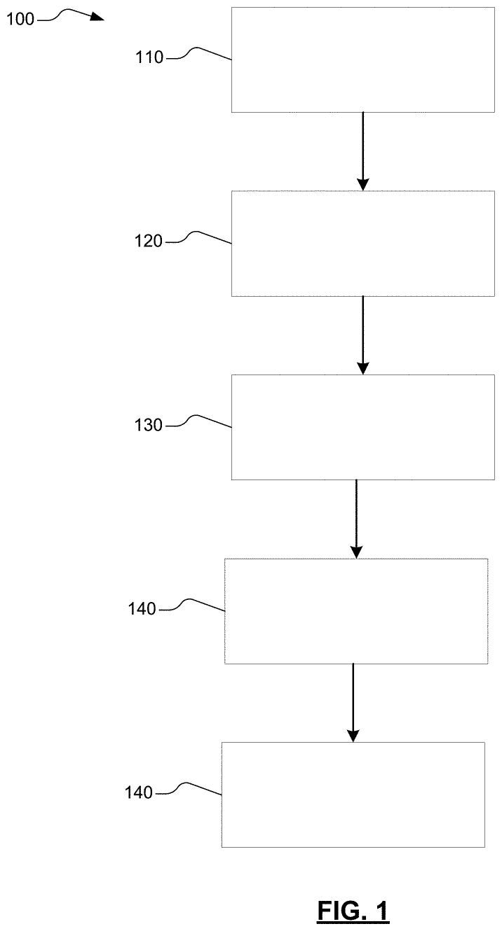

An example method 100 for identifying appropriate and effective treatments for an infection (e.g., SARS-COV-2) is illustrated in FIG. 1. The method 100 for identifying appropriate and effective treatments may generally include (i) identifying or determining 110 affected or involved biological processes; (ii) identifying or determining 120 impacted pathways associated with differentially expressed genes, differentially expressed proteins, and/or similar omics data (such as, methylation, microRNA, metabolomics, and the like); (iii) identifying or determining 130 upstream regulators involved in the infection and/or immunological response to the infection as expressed by the differentially expressed genes, the differentially expressed proteins, and/or the similar omics data (such as, methylation, microRNA, metabolomics, and the like); (iv) identifying or determining 140 involved mechanisms (i.e., mechanisms likely to be involved in the impacted pathways and/or linking genes involved in the key biological processes), and (v) identifying or determining 150 targeted drug treatment (i.e., known drugs capable of reversing the most relevant gene expression differentially expressed proteins, and/or similar omics changes induced by the infection). The present disclosure provides for one or more systems, methods, computation methods, and computer readable media configured to complete one or more of the above steps. For example, the one or more systems may include a controller having a processor and including a display or output.

One or more of the above steps may include providing data. For example, identifying or determining 110 affected or involved biological processes may include providing data on the expression levels of a plurality of biomolecules differentially expressed in a disease state as compared with the same biomolecules expressed in a non-diseased state, as further detailed below.

In certain variations, identifying or determining 100 the affected or involved biological processes (“BP”) may generally include obtaining a plurality of biomolecules (e.g., mRNA, protein, methylation, miRNA, etc.) from both infected and non-infected subjects and calculating differences between the different and determining an applicable p-value for each biomolecule. For example, the affected or involved biological processes (“BP”) may be identified or determined 110 using an enrichment analysis, such as a Gene Ontology (“GO”) analysis, to isolate differentially expressed genes, which are those having over-represented and/or under-represented profiles. For example, a computerized analysis approach may be used to compare a number of over-represented and/or under-represented differentially expressed genes (i.e., biomolecules differentially expressed in a disease state) to a number of differentially expressed genes expected by chance (i.e., same biomolecules expressed in a non-diseased state). In various aspects, the computerized analysis approach may compare the levels of biomolecules in samples affected with COVID19 and the levels of the same type of biomolecules in samples unaffected with the disease (i.e., healthy or control cells). For example, the levels of biomolecule measured may be compared in one or more tissues, such as normal human bronchial epithelial (“NHBE”) cells or whole lung tissue, or cell lines (such as, A549). In order to understand the differences between a first pathogen and another or section pathogen, the levels of the same type of biomolecules may be compared in samples infected with other pathogens, such as influenza A virus (“IAV”) or respiratory syncytial virus (“RSV”) as compared to the respective controls. Further, a comparison between the biological processes, pathways, mechanisms, and drug effects between different contrasts involving, for example, different tissues and/or different pathogens, may be used to distinguish what is specific to a target pathogen or disease as compared to a general response to infection. The same comparison may be used to distinguish the biological processes, pathways, and mechanisms and to recommend drugs that would be specific to a given tissue or cell line, as compared to the characterization of a given disease or pathogen across various tissues and cell types.

In each instance, the computerized analysis approach may then apply a statistical analysis to identify the Gene Ontology categories (e.g., biological processes, molecular functions, cellular locations, etc.) that are over-represented and/or under-represented for the particular condition or disease under study (e.g., using an over-representation approach (“ORA”)). For example, a statistical analysis (e.g., hypergeometric distribution) can be used to calculate the probability of observing the actual number of genes just by chance (i.e., a p-value). The p-value may be corrected using false discovery rate (“FDR”) and/or Bonferroni correction methods. In each instance, the gene ontology analyses may be subject to intelligent prunning approaches, such as elim and weight pruning methods. Such approaches may generally include constructing a custom cut through the gene ontology hierarchy by starting with the most specific nodes and calculating their p-values with all genes assigned directly to each such node, and then, if a node is found to be significant itis reported, and if the node is not significant, the genes associated with the given node are propagated to its direct ancestors and a p-value is calculated for each of the direct ancestors.

In other variations, the affected or involved biological processes (“BP”) may be identified 110 using a functional class scoring (‘FCS”), such as Gene Set Enrichment Analysis (“GSEA”), which includes another computerized analysis approach that ranks all genes based on a correlation between their expressions and given phenotype, and calculates a score that reflects the degree to which a given pathway (P) is represented at the extremes of the entire ranked list. A score may be calculated by walking down the list of genes ordered by expression change. The score may be increased for every gene that belongs to the given pathway (P) and decreased for every gene that does not. Statistical significance (i.e., a p-value) can be established using a null distribution constructed by permutations.

In each instance, the input data relating to the biological process(es) may come from human subjects or non-human animal subjects, including non-human mammals, birds, reptiles, amphibians, and fish. Non-limiting examples of non-human mammals includes dogs, cats, cattle, and bats.

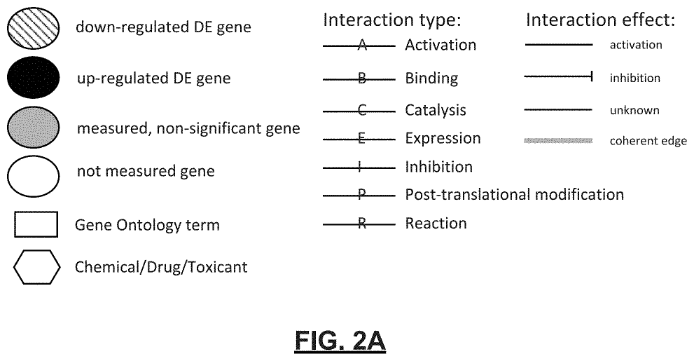

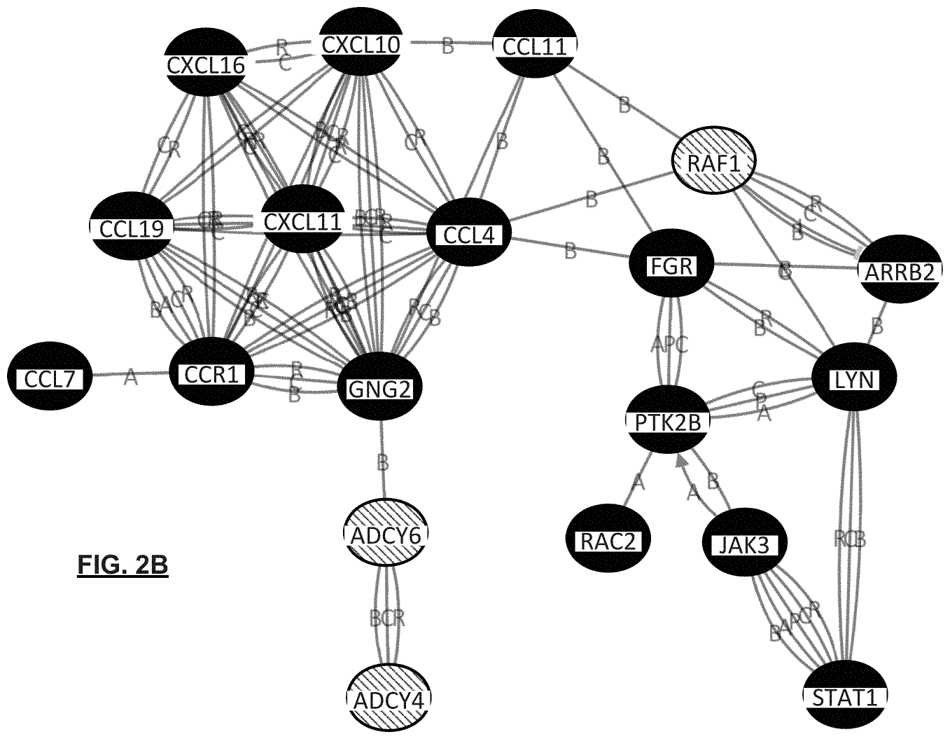

In certain variations, the impacted pathway may be identified 120, for example, using impact analysis methods that consider both (a) the over-representation and/or under-representation of differentially expressed genes in a given pathway (i.e., enrichment) and (b) the perturbation of the given pathway computed by propagating the measured expression changes across the pathway topology, such as obtained from the Kyoto Encyclopedia of Genes and Genomes (“KEGG”) database. For example, FIGS. 2A and 2B illustrate the differentially expressed genes on a chemokine signaling pathway.

The over-representation and/or under-representation of the differentially expressed genes in a pathway may be represented by a first probability value (e.g., pORA). The perturbation of the given pathway may be represented by a second probability value (e.g., pACC). The first probability (pORA) expresses the probability obtaining a number of differentially expressed genes in a given pathway that is greater than or equal to the actually observed number of DE genes, just by chance. Additional details and/or alternatives for the impact analysis method, including additional details and/or alternatives for determining the first probability (pORA) and/or the second probability (pACC) may be found in U.S. application Ser. No. 12/180,303 titled Method for Analyzing Biological Network and issued as U.S. Pat. No. 8,068,994 on Nov. 29, 2011.

The second probability (pACC) can be calculated using an amount of total accumulation measured in each pathway. For example, a perturbation factor may be computed for each gene on the pathway using Formula I:

$$PF ( g ) = α ( g ) \cdot Δ E ( g ) + {\sum }_{u ∈ {US}_{g}} {β}_{ug} \frac{PF ( u )}{{N}_{ds} ( u )} \qquad ( Formula Ι )$$

where PF(g) is the perturbation factor for gene (g), ΔE(g) represents the observed fold change of gene (g), and α(g) is a priori weight used to incorporate information about the type of gene or its significance. In certain variations, all genes may be treated equally, such that (g)=1, for all genes (g). The last term (i.e., the sum term) is the sum of the perturbations coming from all genes (u) situated upstream of gene (g), where US_{g} denotes all genes upstream of the gene (g). The perturbation coming from the upstream genes (u), and affecting gene g, can be calculated as the sum of all perturbation factors of all genes (u) normalized by the number of downstream genes of each such gene N_{ds}(u) and multiplied by β_{ug}, which quantifies the strength of the interaction between genes (g) and all genes (u), where the sign of β represents the type of interaction (e.g., positive for activation-like signals and negative for inhibition-like signals). The accumulation for each gene (g) level may be represented by Acc(g), which is the difference between the perturbation factor (PF(g)) and the observed fold change as presented in Formula II:

Acc(g)=PF(g)−ΔE(g) (Formula II)

The perturbation accumulations for all genes on a pathway may be simultaneously computed by solving the system of linear equations as resulting from the combination of Formula I for each gene (g). Once the perturbation accumulations are computed, the total accumulation of the pathway (pACC) may be calculated as the sum of all absolute accumulations of genes in a given pathway.

The first and second probabilities may be combined for determined a unique pathway-specific uncorrected p-value, for example, using the Fisher's method. The unique pathway-specific uncorrected p-value may be corrected for multiple comparisons using false discovery rate (“FDR”) and Bonferroni corrections.

In certain variations, identifying the acting or involved upstream regulators may be based on, for example, (a) the over-representation and/or under-representation of differentially expressed genes (i.e., enrichment) and (b) network of known interactions, such as from the Advaita Knowledge Base (“AKB”). The network may be a directed graph in which a source node represents either a chemical substance or compound, a drug, or a toxicant. In such instances, the edges represent known effects that the chemical substance or compound, a drug, or a toxicant has on various genes, while a signed edge includes a source chemical substance or compound, a drug, or a toxicant (u), a target gene, and a sign to indicate the type of effect (e.g., activation (+1) or inhibition (−1)). As noted, in this instance, the variable u stands for the chemical substance or compound, a drug, or a toxicant. However, its role here is similar to the role of the upstream gene as used above in Formula I. In both instances, the analysis is performed on a graph in which u is the node immediately upstream. In the first instance, the upstream node represents a gene, while here the upstream node represents the chemical substance or compound, a drug, or a toxicant.

In certain variations, the involved mechanisms (i.e., potentially active mechanisms on existing pathways) may be identified 140, for example, using putative mechanism inference based on the pathways and biological processes identified, as well as the measured fold changes in genes, proteins, or other biomolecules participating in the identified pathways and biological processes and all known protein-protein interactions (“PPIs”), for example, both from existing pathways and known protein-protein interactions databases (such as, STRING). In various aspects, putative mechanisms may be identified as sequences of pathway signals for which the measured gene expression changes are consistent with the sequence of events described by the pathway. For example, considering gene A which is known to activate or upregulate gene B, where gene B is immediately downstream of gene A. As such, when gene A is downregulated, gene B will be expected to also be downregulated; and if gene A is upregulated, gene B will be expected to be upregulated as well. Conversely, if gene A is known to down-regulate gene B, then when gene A is upregulated, gene B would be expected to be upregulates; and if gene A is downregulated, then gene B would be expected to be downregulated. Thus, if the measured values of gene B are consistent with the expectations created by the measured value of gene A and the type of edge between gene A and gene B, then the edge between gene A and gene B, representing a signal from A to B, is said to be “consistent”. If, for instance gene A downregulates B and gene A is upregulated and gene B is also upregulated, the corresponding edge will not be consistent with the expectations of the known phenomena, as described by the pathway. Putative mechanisms represent a sequence of two or more of such consistent edges.

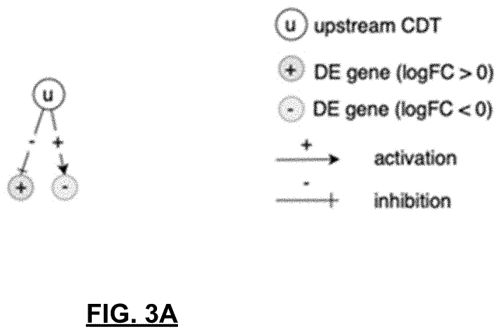

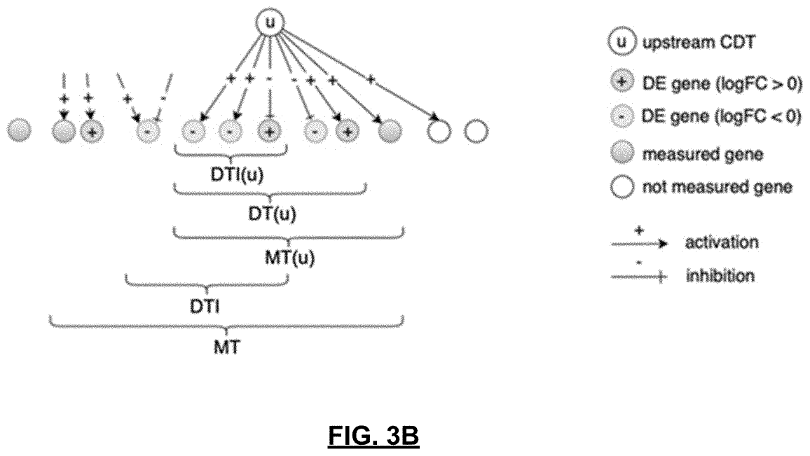

In certain variations, a drug-target analysis may be used to estimate 150 the ability of chemicals (e.g., FDA-approved drugs, drug candidates, or other chemicals) to substantially reverse one or more of the most relevant gene expression changes (i.e., observed differentially expressed genes) induced by the disease. That is, a drug-target analysis may be used to identify 150 likely upstream chemicals, drugs, and/or toxicants (“CDTs”) that can substantially reverse one or more of the most relevant gene expression changes induced by the disease. For example, as illustrated in FIG. 3A, the chemical substance or compound, drug, or toxicant (u) may repress the gene which is upregulated by the infection and activated the gene which is repressed.

Identifying 150 an effective drug treatment may include (a) identifying the number of differentially expressed genes that would be reverted, as well as (b) determining a p-value (e.g., a Bonferroni-corrected p-value) that indicates the likelihood of having the observed number of reversible genes just by chance, where small p-values denote observations that are unlikely to be a result of only random change. Suitable drugs have both small p-values (i.e., lower than a predetermined threshold of significance (such as, about 0.1, about 0.05, or about 0.01)) and revert a large number of differentially expressed genes. For example, FIG. 3B illustrates all measured target genes (represented by “MT”), including a subset of differentially expressed targets (e.g., differentially expressed genes, differentially expressed proteins, and/or similar omics data (such as, methylation, microRNA, metabolomics, and the like)) consistent with the hypothesis that the chemical substance or compound, drug, or toxicant (u) are insufficient (represented by “DTI”), and for the selected chemical substance or compound, drug, or toxicant (u), the measured targets of the upstream chemical substance or compound, drug, or toxicant (u) are represented as MT(u), the differentially expressed gene targets downstream of u are represented as DT(u), and the set of differentially expressed gene targets consistent with the hypothesis that u is insufficient is represented by DTI(u).

To identifying the number of differentially expressed genes, the drug-target analysis may consider the hypothesis that the chemical substance or compound, drug, or toxicant (u) is absent (or insufficient) in the condition. That is, a drug for which the hypothesis is supported by a significant amount of evidence will be a very strong candidate for repurposing because such chemical substance or compound, drug, or toxicant (u) will effectively reverse many of the gene expression changes observed as a result of the disease. A gene may be a target gene of the chemical substance or compound, drug, or toxicant (u) if it is known that the given chemical changes the expression level of the given gene (g). A consistent gene is a target gene that is differentially expressed and the sign of the expression change is consistent both with the type of the signal and with the hypothesis considered. For example, if the chemical is known to increase the expression of the gene, and the hypothesis of an insufficient amount of chemical being present is considered, the genes that are known to be affected by the chemical that are measured to be downregulated will be consistent with this hypothesis, while all genes known to be downregulated by the chemical and measured and found to be upregulated will also be consistent with the hypothesis.

A p-value that indicates the suitability of the proposed drug treatment can be calculated as a combination of a first probability (P_{abs}) and a second probability (P_{z}), and the method 100 may include determining a first probability (P_{abs}) and a second probability (P_{z}) and combining the first and second probabilities using a Fisher's method. For example, for each upstream chemical substance or compound, drug, or toxicant (u) hypothesized to be insufficient, the number of consistent differentially expressed genes downstream of the upstream chemical substance or compound, drug, or toxicant (u) (represented by DTI(u)) is compared to the number of measured target genes expected to be both consistent and differentially expressed by change. An uncorrected p-value (i.e., the first probability (P_{abs})) is computed using a hypergeometric model or similar (e.g., chi-square, Fisher's exact test, Bayesian, etc.)

For the hypothesis (i.e., that the chemical substance or compound, drug, or toxicant (u) is absent (or insufficient) in the condition), the drug-target analysis may compute a z-score (z(u)) for each chemical substance or compound, drug, or toxicant (u) and a p-value corresponding to the z-score (P_{z}) may be the second probability. In certain variations, the drug-target analysis may compute a z-score (z(u)) by iterating over the genes that are differentially expressed and immediately downstream of the chemical substance or compound, drug, or toxicant (u) as shown in Formula III:

$$z ( u ) = \frac{{\sum }_{g ∈ DT ( u )} sign ( e ( u , g ) ) \times sign ( g )}{\sqrt{\vert DT ( u ) \vert}} \qquad ( Formula ΙΙΙ )$$

where sign(g) is the sign of the differentially expressed gene (g) (e.g., +1 for upregulated and −1 for down regulated), e(u, g) represents an edge from u tog, sign(e(u,g)) represents the type of interaction (e.g., +1 for activation and −1 for inhabitation) and |DT(u)| represents the number of genes that are differentially expressed and immediately downstream of the chemical substance or compound, drug, or toxicant (u). In certain variations, a second probability may also be calculated using other similar statistical models.

Under the null hypothesis, the sign of an edge (sign(eᵢ)) and the sign of a gene (sign(gᵢ)), may be independent and identically distributed random variables that have either values −1 or +1, and where Xᵢ represents the product of the two variables (i.e., Xᵢ=sign(eᵢ)×sign(gᵢ)). As such, Xᵢ is a random variable taking values from {−1,1}, with the expected value E[Xᵢ]=μ=0 and variance Var[Xᵢ]=σ²=1. Considering an upstream chemical substance or compound, drug, or toxicant (u) with a number (n) of downstream genes (i.e., n=|DT(u)|), the average number of n samples of Xᵢ, i∈{1, . . . , n} is Xₙ. As such, according to the central limit theorem, the variables n(Xₙ−μ) converge for a normal distribution N(0,1) for large values of n, where μ=0, may be represented as shown in Formula IV:

$$\sqrt{n} \cdot {\overline{X}}_{n} = \frac{n \cdot {\overline{X}}_{n}}{\sqrt{n}} = \frac{n \cdot \frac{{\sum }_{i = 1}^{n} {X}_{i}}{n}}{\sqrt{n}} = \frac{{\sum }_{i = 1}^{n} {X}_{i}}{\sqrt{n}} \qquad ( Formula ΙV )$$

where n=|DT(u)|, and as illustrated, the numerator may be the sum of all n samples Xᵢ, which is also the numerator as illustrated in Formula III. As such, the variable z(u) follows a standard normal distribution (N(0,1)), and the p-value as corresponding to the z-score (P_{z}) as the one-tail area under the probability density function for the normal distribution.

The present disclosure also provides methods for treating infections or disorders using treatments identified, for example, using the method 100 as illustrated in FIG. 1. In certain variations, methods for treating infections or disorders may include treating physiologies and symptoms associated with a corona virus in a subject in need thereof, or other infections agents associated with aberrant immune responses. For example, the present disclosure provides methods for treating local or systemic hyperinflammation, including, for example, an aberrant immune response and cytokine storms, as associated with corona virus (e.g., corona virus disease 2019 (COVID-19), severe acute respirator syndrome coronavirus 2 (SARS-CoV-1), Middle East respiratory syndrome corona virus (MERS-CoV), and severe acute respirator syndrome coronavirus 2 (SARS-CoV-2), and the like), severe acute respiratory syndrome (“SARS”), pneumonia, acute respiratory distress syndrome (“ARDS”), septic shock, multi-organ failure, and combinations thereof as non-limiting examples. In each instance, the treatment includes at least one of alleviating the disorder or alleviating symptoms associated with the disorder in the subject, and the subject can be a human or non-human animal.

A method for treating infections or disorders using treatments identified, for example, using the method 100 as illustrated in FIG. 1, may include administering to a subject a therapeutically effective amount of a composition. The composition may include one or more compounds selected from the group consisting of: methylprednisolone, tofacitinib, dexamethasone, prednisolone, diclofenac, myochrysine, esters thereof, derivatives thereof, salts thereof, and combinations thereof, where each compound can decrease inflammation in subjects with certain indications. Myochrysine is also referred to as gold sodium thiomalate, and where methylprednisolone and prednisolone are corticosteroids, diclofenac is a nonsteroidal anti-inflammatory drug (“NSAID”), and tofacitnib and myochrysine are antirheumatics. The administering may be performed orally, sublingually, intravenously, intramuscularly, or subcutaneously.

In each instance, “therapeutically effective amount” refers to an amount of the compound that, when administered to the subject having the disorder or suspected of having the disorder, is sufficient, either alone or in combination with additional therapies, to effect treatment for the disorder. The “therapeutically effective amount” is expected to vary depending on, for example, the compound, pharmaceutical composition or pharmaceutical dosage form, the disorder treated and its severity, and the age and weight of the patient to be treated.

In each instance, the composition can be administered in a single dose or divided between a plurality of doses each day, as directed by the care provider. For example, the treating may include administering an initial dose(s) to the subject, which is follow by regular scheduled dosing. The initial dose may be a loading dose, (i.e., a dose that is more concentrated than the following doses) or a test dose (i.e., a dose that is less concentrated than the following doses and intended to be used to determine if any negative side effects result in the subject from the test dose).

In certain variations, the one or more compounds may include methylprednisolone, an ester thereof, a derivative thereof, or a salt thereof (collectively “methylprednisolone”) to the subject, and the administering may include administering a therapeutically effective amount of the methylprednisolone composition to the subject. In some example embodiments, a therapeutically effective amount of the methylprednisolone composition may include the methylprednisolone at a dosage of greater than or equal to about 0.1 mg to less than or equal to about 300 mg per day, optionally greater than or equal to about 0.1 mg to less than or equal to about 200 mg per day, or in certain aspects, optionally greater than or equal to about 40 mg to less than or equal to about 50 mg. However, the dosage may be modified by the care provider, for example, because of the age and/or weight of the subject.

In certain variations, the one or more compounds may include prednisolone, an ester thereof, a derivative thereof, or a salt thereof (collectively “prednisolone”) to the subject, and the administering may include administering a therapeutically effective amount of the prednisolone composition to the subject. In some example embodiments, a therapeutically effective amount of the prednisolone composition may include the prednisolone at a dosage of greater than or equal to about 0.1 mg to less than or equal to about 300 mg per day, or in certain aspects, optionally greater than or equal to about 0.1 mg to less than or equal to about 200 mg per day. However, the dosage may be modified by the care provider, for example, because of the age and/or weight of the subject.

In certain variations, the one or more compounds may include diclofenac, an ester thereof, a derivative thereof, or a salt thereof (including potassium and sodium salts) (collectively “diclofenac”) to the subject, and the administering may include administering a therapeutically effective amount of the diclofenac composition to the subject. In some example embodiments, a therapeutically effective amount of the composition includes the diclofenac at a dosage of greater than or equal to about 1 mg to less than or equal to about 225 mg per day. However, the dosage may be modified by the care provider, for example, because of the age and/or weight of the subject.

In certain variations, the one or more compounds may include myochrysine, an ester thereof, a derivative thereof, or a salt thereof (collectively “myochrysine”) to the subject, and may include administering a therapeutically effective amount of the myochrysine composition to the subject. In some example embodiments, a therapeutically effective amount of the composition includes the myochrysine at a dosage of greater than or equal to about 1 mg to less than or equal to about 1000 mg per week, with the proviso that no single dose includes more than about 100 mg of myochrysine. For example, a first weekly dose can include about 10 mg myochrysine and additional weekly doses can include about 25 mg myochrysine. However, in each instance, the dosage may be modified by the care provider, for example, because of the age and/or weight of the subject.

In various aspects, the method for treating infections or disorders using treatments identified, for example, using the method 100 as illustrated in FIG. 1, may include administering (i) a therapeutically effective amount of a first composition and (ii) a therapeutically effective amount of a second composition to a subject. The first composition may include, for example, one or more first compounds selected from the group consisting of: prednisolone, dexamethasone, diclofenac, myochrysine, esters thereof, derivatives thereof, salts thereof, and combinations thereof. The second composition may include, for example one or more second compounds selected from the group consisting of: methylprednisolone, tofacitinib, prednisolone, dexamethasone, diclofenac, myochrysine, esters thereof, derivatives thereof, salts thereof, and combinations thereof, where the second composition is different from the first composition. The first and second compounds may be administered concurrently (i.e., coadministering) or subsequently.

In various aspects, the method for treating infections or disorders using treatments identified, for example, using the method 100 as illustrated in FIG. 1, may include coadministering (i) a therapeutically effective amount of a composition and (ii) an adjunct therapeutic agent to a subject. In certain variations, the composition may include one or more compounds selected from the group consisting of: methylprednisolone, tofacitinib, prednisolone, dexamethasone, diclofenac, myochrysine, esters thereof, derivatives thereof, salts thereof, and combinations thereof, where each compound can decrease inflammation in subjects with certain indications. In other variations, the composition may include one or more compounds selected from the group consisting of: prednisolone, dexamethasone, diclofenac, myochrysine, esters thereof, derivatives thereof, salts thereof, and combinations thereof. In each instance, the adjunct therapeutic agent may be an inti-inflammatory agent, such as a corticosteroid, a steroid, a nonsteroidal anti-inflammatory drug (“NSAID”), an antirheumatic, an anti-parasitic agent, an anti-bacterial agent, an anti-viral agent, or combinations thereof, as non-limiting examples. The coadministering may be performed simultaneously with or separately from the administering of the composition.

Certain features of the current technology are further illustrated in the following non-limiting example.

### Example 1

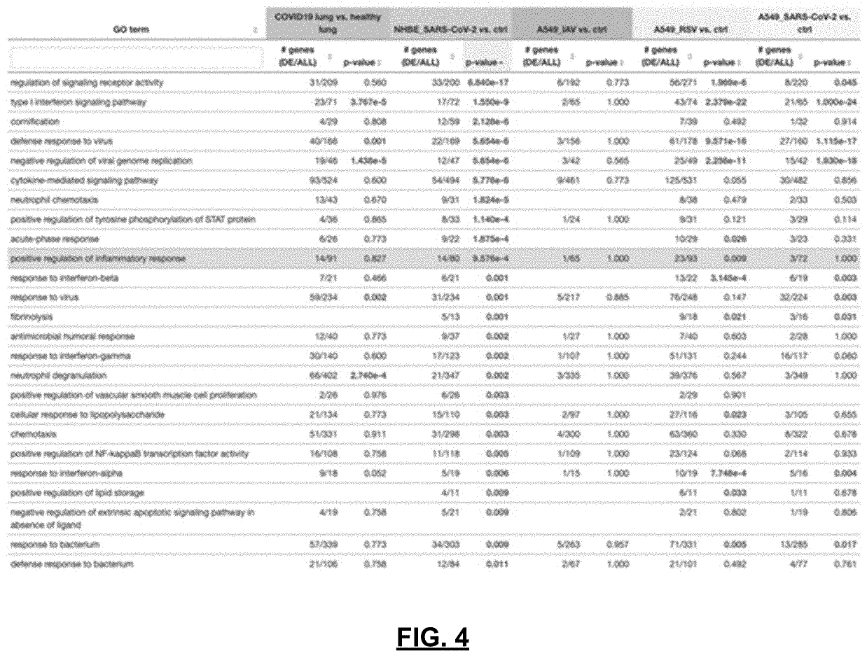

Various biological processes were evaluated for infected cells and control cells. For example, biological processes were compared for (a) COVID19 v. Control, (b) NHBECOV2 v. Control, (c) A549CoV2 v. Control, (d) A549IAV v. Control, and (e) A549RsV v. Control to identify over-represented and/or under-represented differentially expressed genes. As illustrated in FIG. 4, although there was a larger number (815) of differentially expressed genes in the SARS-Cov-2-infected lung, there were only seven significant biological processes identified, which suggests a more coordinated, systemic response. In contrast, the changes in the NHBE cells are characterized by fewer differentially expressed genes (223), but span more uncoordinated biological processes. In FIG. 4, the rows are ordered by their significance in NHBE-CoV2 v. Control. In FIG. 4, the columns titled “#genes” represent the number of differentially expressed genes out of number of genes associated with the given biological process.

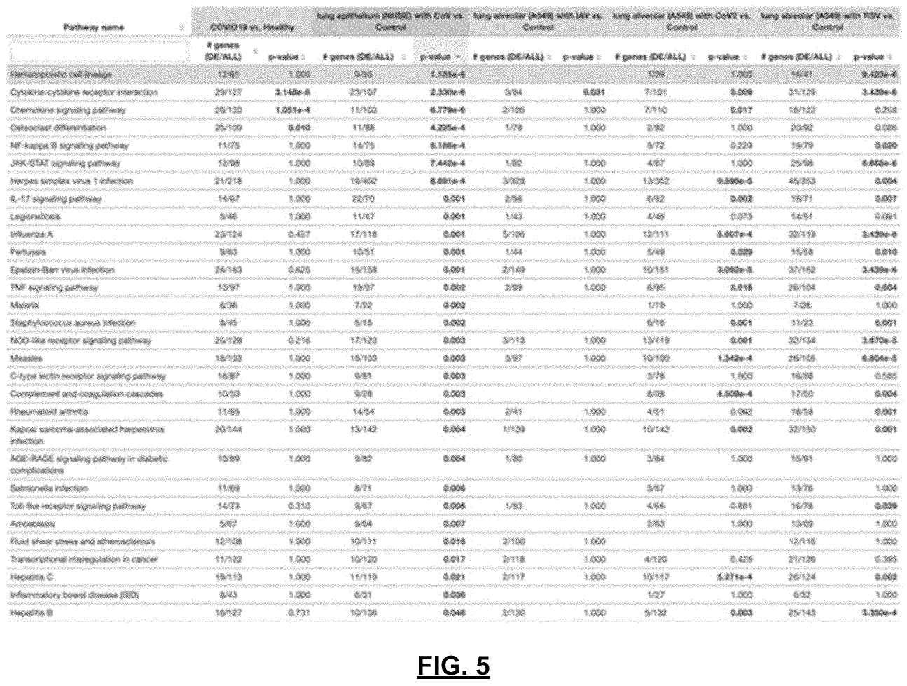

Impacted pathways for the differentially expressed genes were then identified using impact analysis methods like those detailed above. For example, FIG. 4 illustrates the signaling pathways in ordered by their significance in NHBE-CoV2 v. Control. As in FIG. 4, the columns titled “#genes” in FIG. 5, represent the number of differentially expressed genes out of number of genes associated with the given biological process. As illustrated in FIG. 5, the most impacted pathway in the NHBE-CoV2 v. Control is the hematopoietic pathway, which may be linked to the hyper-coagulation phenomena observed in many COVID-19 patients, and the most impacted pathways in the COVID19 v. Control is the cytokine-cytokine interactions. Upon study of the cytokine-cytokine receptor interactions pathway, and based on the observed changes in their downstream genes, four genes were identified as activated upstream regulators including (i) IRF9, (ii) STAT2, (iii) IFNG, and (iv) IFNB1, which suggests two different potential mechanisms. The first mechanism is triggered by STAT2 and IRF9 and has sixteen common target genes that are also all significantly upregulated, including IFI6, IFIT1, IFIT2, IFIT3, IFITM1, IFITM3, OAS1, OAS3, OAS S32, MX1, MX2, RSAD2, OASL, XAF1, IRF2, and IRF7, which are inducers of apoptosis (XAF1, IRF2, IRF7). The first mechanism is known to be involved in the response to influenza A.

The second mechanism involves interferon beta and gamma, which target five downstream genes, including CXCL10, IDO1, DOX58, STAT1, which are upregulated, and HMOX2 which is downregulated. CXCL10, IDO1, DOX58, and STAT1 are genes associated with lymphocyte recruitment and immune regulation. Interferon regulatory factors (“IRFs”) are subdivided into the interferonic IRFs (IRF2-3-7 and 9), the stress responsive IRFs (IRF1 and 5), the hematopoietic IRFs (IRF4 and 8), and morphogenic IRF6. IRF9 is a regulator of type I IFN signaling and is known to interact with STAT2 and STAT1 to form the heterotrimeric transcription factor complex (ISGF3) that binds to interferon-stimulated response elements (ISREs) to induce the expression of interferon stimulated genes (ISG). During viral infections, ISGs perform two key functions: (a) directly limit viral replication by shutting down protein synthesis and triggering apoptosis, and (a) activate key components of the innate and adaptive immune system, including antigen presentation and production of cytokines.

Genes known to modulate or inhibit the inflammatory response (such as, IL1RN IL10, and IL13) were also considered. In the COVID19vsControl, IL1RN was up with a log 2 fold change of 6.2 fold (a 78-fold increase, FDR-corrected p=10⁻⁶), IL10 was up 2.8 fold (FDR-corrected p=0.55), while the measurement for IL13 was not available. In the NHBECoV2vsControl, IL1RN was up only 1.26 fold (FRD-corrected p=0.035), while measurements for IL10 and IL13 were not available. However, in this contrast, fourteen out of fifteen DE genes immediately downstream of IL10, and usually inhibited by IL10, were upregulated which strongly supports the hypothesis that IL10 is inhibited (FDR-corrected p=5.17×10⁻⁹). Together, the GO analysis, pathway analysis and the putative mechanisms identified by the analysis above strongly suggest a hyperinflammation/cytokine storm.

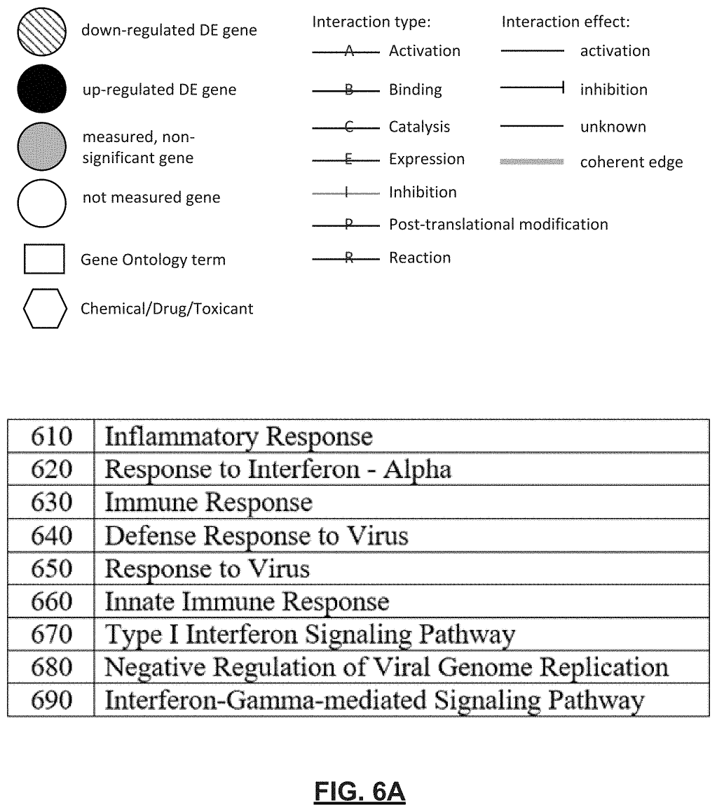

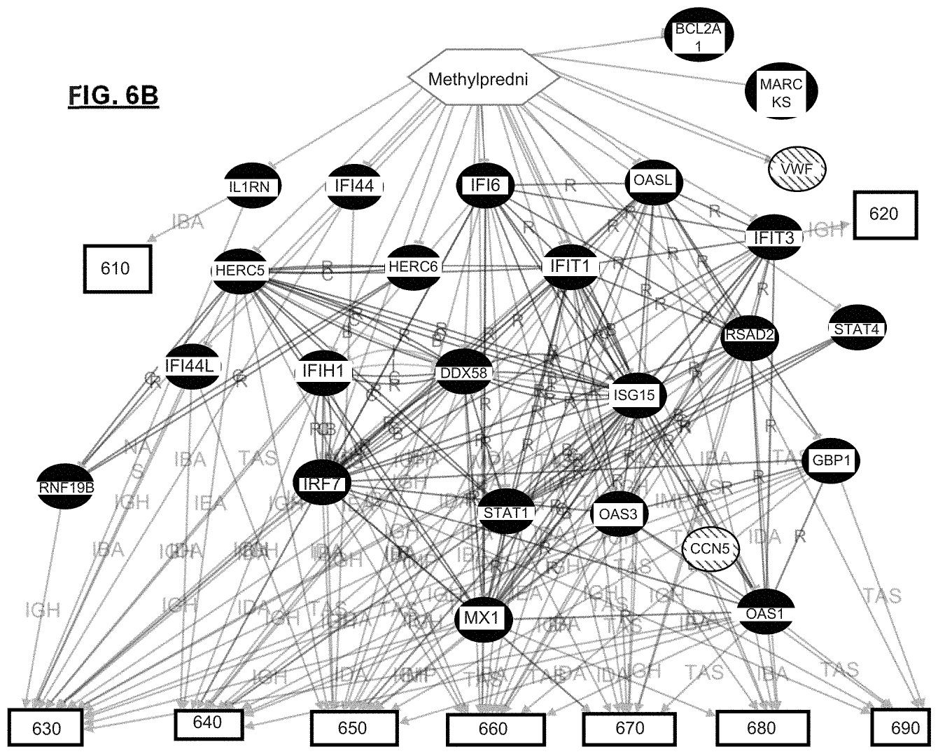

Once the main regulatory pathways potentially associated with hyper-inflammation were identified, targeted drug treatment were identified. For example, using the method detailed above, including the number of differentiating expressed genes and the p-value (e.g., Bonferroni-corrected p-value) indicating the suitability of each proposed drug for repurposing in COVID-19, FDA-approved drugs that could show activity on multiple components of inflammation were considered for use in the management of severe COVID-19 cases. In particular, suitable drugs were those having both small p-values and revert a large number of differentially expressed genes. Five possible drugs were identified for the use in the management of severe COVID-19 cases, including (i) methylprednisolone (“MP”) and (ii) prednisolone, which are corticosteroids currently used to modulate the immune response in rheumatoid arthritis, (iii) diclofenac a non-steroidal anti-inflammatory drug (“NSAID”), (iv) tofacitinib a JAK inhibitor, and (v) gold sodium thiomalate (i.e., myochrysine) an older anti-inflammatory drug, also used in the treatment of rheumatoid arthritis. For example, methylprednisolone targets twenty-seven genes that are found to be differentially expressed in the COVID19 vs. control comparison. Out of these twenty-seven genes, the drug would revert the changes in twenty-five of them. Methylprednisolone also had an extremely significant p-value even after a Bonferroni correction, which is the most stringent correction available (p=5.72×10⁻¹⁰). Methylprednisolone also reverts twenty-two out of twenty-two genes found to be differentially expressed in NHBECoV2vsControl, and twenty-five out of twenty-six genes found to be differentially expressed in A549CoV2vsControl. For example, FIGS. 6A and 6B illustrate the putative mechanism through which methylprednisolone acts on the identified differentially expressed genes, and also, how those differentially expressed genes influence the identified biological processes.

Notably, drugs in the same class might not necessarily have similar effects. For example, methylprednisolone and prednisolone, corticosteroids currently used to modulate the immune response in rheumatoid arthritis, are effective in reverting many of the changes triggered by COVID-19, while other closely-related corticosteroids (such as, prednisone) were not predicted to be equally effective. The putative mechanisms through which the other closely-related corticosteroids (such as, prednisone and/or hydrocortisone) would revert the genes dysregulated in COVID-19 are different. Methylprednisolone inhibits STAT1, IFT3 and HERC5, and prednisolone has an impact on IFIT genes (such as, IFT1, IFIT3, IFI6, and IFI4L), while prednisone targets only three differentially expressed genes in the COVID19 vs Control and only two differentially expressed genes in the NHBECoV2 vs Control. Prednisone only reverts only one of the three differentially expressed genes in the COVID19 vs Control and zero out of two differentially genes in the NHBECoV2 vs Control.

Further, though prednisolone, dexamethasone, and hydrocortisone belong to the same family of corticosteroid anti-inflammatory agents and include structural similarities, hydrocortisone reverts only eight out of ten differentially expressed genes in the COVID19 vs Control (e.g., FDR-corrected p=0.57) and five out of eight differentially expressed genes in the NHBECoV2 vs Control (e.g., FDR-corrected p=0.038, Bonferroni-corrected p=1); dexamethasone reverts only thirty-three out of sixty-nine differentially expressed genes in the COVID19 vs Control (e.g., FDR-corrected p=1) and twenty-seven out of forty-five differentially expressed genes in the NHBECoV2 vs Control (e.g., FDR-corrected p=0.002, Bonferroni-corrected p=0.066); and dexamethasone is significant in the NHBECoV2 vs Control but not in the COVID19 vs Control. Further, hydrocortisone appears as significant in COVID19 vs Control, but only marginally so in the NHBECoV2 vs Control.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A method for identifying targeted treatments for treatment of a phenotype in a human or non-human subject, the method comprising:
providing data relating to a plurality of affected biomolecules to one or more processors;
the one or more processors, using a first probability, identifying one or more biological pathways impacted be the affected biomolecules;
the one or more processors, identifying one or more upstream regulators in each of the one or more biological pathways; and
the one or more processors, using a second probability, determining at least one targeted treatment configured to reverse expressions of at least one of the affected biomolecules and the one or more biological pathways.

2. The method of claim 1, wherein the data relating to the plurality of affected biomolecules comprises p-values for each of the affected biomolecules, and wherein the method further comprises:
the one or more processors, obtaining a first data collection relating to a first plurality of biomolecules in affected subjects and a second data collection relating to the first plurality of biomolecules in unaffected subjects; and
the one or more processors, using the first data collection and the second state collection, determining p-values for each of the affected biomolecules.

3. The method of claim 1, wherein the method further comprises:
preparing the data relating to the plurality of affected biomolecules, wherein the data is prepared by comparing first expression levels of a plurality of expressed genes in affected subjects and second expression levels of the plurality of expressed genes in unaffected subjects.

4. The method of claim 1, wherein the data relating to the plurality of affected biomolecules defines a plurality of differentially expressed biomolecules.

5. The method of claim 4, wherein the first probability comprise:
a first probability value (pORA) that is a probability of observing a number of the differentially expressed biomolecules in a respective biological pathway of the one or more biological pathways just by chance; and
a second probability value (pACC) that is the probability of observing a total perturbation accumulation as measured in each of the respective biological pathways of the one or more biological pathways just by chance.

6. The method of claim 5, wherein the method further comprises:
combining the first probability value (pORA) and the second probability value (pACC) so as to determine a unique pathway-specific uncorrected p-value for each of the one or more biological pathways.

7. The method of claim 6, wherein the targeted treatment has a p-value that is less than a predetermined threshold of significance and reverses expression of more than two of the respective differentially expressed biomolecules.

8. The method of claim 1, wherein the method further comprises, prior to the identifying of the targeted treatment:
the one or more processors, using a putative mechanism inference approach, determining mechanisms involved within the respective biological pathway of the one or more biological pathways and linking one or more of the plurality of differentially expressed genes.

9. The method of claim 8, wherein the putative mechanism inference is further based on at least one of measured fold changes in the respective biomolecules of the plurality of biomolecules or the respective biological pathways of the one or more biological pathways and known protein-protein interactions (“PPIs”) for the respective biological pathways of the one or more biological pathways.

10. The method of claim 1, the method further comprising:
treating an infection associated with a corona virus in a human or non-human subject, wherein the treating comprises:
administering to the subject a therapeutically effective amount of a composition comprising a compound selected from the group consisting of: prednisolone, dexamethasone, diclofenac, myochrsine, esters thereof, derivatives thereof, salts thereof, and combinations thereof.

11. The method of claim 10, wherein the composition is a first composition and the compound is a first compound, and the treating further comprises:
administering to the subject a therapeutically effective amount of a second composition comprising a second compound, wherein the second compound is selected from the group consisting of: methylprednisolone, tofacitinib, dexamethasone prednisolone, diclofenac, myochrysine, esters thereof, derivatives thereof, salts thereof, and combinations thereof, wherein the second composition is different from the first composition and the administering to the subject the therapeutically effective amount of a second composition is performed simultaneously with or separately from the administering of the first composition.

12. The method of claim 10, wherein the treating further comprises:
coadministering an adjunct therapeutic agent to the subject, wherein the coadministering is performed simultaneously with or separately from the administering the composition.

13. The method of claim 10, wherein the compound comprises prednisolone, an ester thereof, a derivative thereof, or a salt thereof, and the administering of the composition comprises administering to the subject a dosage of greater than or equal to about 0.1 mg to less than or equal to about 300 mg per day.

14. The method of claim 10, wherein the compound comprises diclofenac, an ester thereof, a derivative thereof, or a salt thereof, and administering of the composition comprises administering to the subject a dosage of greater than or equal to about 1 mg to less than or equal to about 250 mg per day.

15. The method of claim 10, wherein the compound comprises myochrysine, an ester thereof, a derivative thereof, or a salt thereof, and administering of the composition comprises administering to the subject a dosage of greater than or equal to about 1 mg to less than or equal to about 1 g per week.

16. A method for identifying targeted drug treatments for treatment of a phenotype in a human or non-human subject, the method comprising:
one or more processors identifying one or more biomolecules affected by the phenotype by isolating a plurality of biomolecules differentially expressed between the phenotype and a control;
the one or more processors, using one or more first probabilities, identifying one or more biological pathways associated with each of the differentially expressed biomolecules;
the one or more processor, identifying one or more upstream regulators;
the one or more processors, using a putative mechanism inference approach, determining mechanisms involved with the respective biological pathway of the one or more biological pathways or linking one or more of the plurality of differentially expressed biomolecules; and
the one or more processors, using one or more second probabilities, determining at least one targeted drug treatment configured to reverse expressions of at least two of the biomolecules that are differentially expressed in the given phenotype.

17. The method of claim 16, wherein using the one or more second probabilities comprises determining a p-value indicative of the chance likelihood of reversing the expressions of the biomolecules the one or more biological pathways, and
wherein the targeted drug treatment has a p-value that is less than a predetermined threshold of significance.

18. The method of claim 16, wherein the method further comprises:
treating an infection associated with a corona virus in a human or non-human subject, wherein the treating comprises:
administering to the subject a therapeutically effective amount of a composition comprising a compound selected from the group consisting of: methylprednisolone, tofacitinib, dexamethasone prednisolone, diclofenac, myochrysine, esters thereof, derivatives thereof, salts thereof, and combinations thereof.

19. A method for identifying targeted drug treatments for treatment of a phenotype in a subject, the method comprising:
one or more processors identifying one or more biomolecules affected by the phenotype by isolating a plurality of differentially expressed genes associated with each of the one or more biomolecules;
the one or more processors determining a first probability value (pORA) that is a probability of observing one or more of the plurality of differentially expressed genes in a biological pathway;
the one or more processors determining a second probability value (pACC) that reflects a total accumulation of the plurality of differentially expressed genes as measured in the biological pathway;
the one or more processors combining the first probability value (pORA) and the second probability value (pACC) so as to determine a unique pathway-specific uncorrected p-value for the biological pathway;
the one or more processors identifying one or more upstream regulators, wherein the one or more upstream regulators are upstream of a respective biomolecule;
the one or more processors, using a putative mechanism inference approach, determining mechanisms involved with the biological pathway or linking one or more of the plurality of differentially expressed genes; and
the one or more processors, using one or more second probabilities, determining at least one targeted drug treatment configured to reverse expressions of at least two target biomolecules found to be differentially expressed in the give phenotype.

20. The method of claim 19, wherein the controller comprises a display, and the method further comprises:
displaying at least one of (i) the one or more biomolecules, (ii) the plurality of differentially expressed genes associated with each of the one or more biomolecules, (iii) the first probability value (pORA), (iv) the second probability value (pACC), (v) the unique pathway-specific uncorrected p-value for the biological pathway, (vi) the one or more upstream regulators in the biological pathway, (vii) the mechanisms involved with the biological pathway or linking one or more of the plurality of differentially expressed genes, (viii) the targeted drug treatment, and (ix) processes thereof.

21. The method of claim 19, wherein the method further comprises:
treating an infection associated with a corona virus in a human or non-human subject, wherein the treating comprises:
administering to the subject a therapeutically effective amount of a composition comprising a compound selected from the group consisting of: methylprednisolone, tofacitinib, dexamethasone prednisolone, diclofenac, myochrysine, esters thereof, derivatives thereof, salts thereof, and combinations thereof.

22. A software program, stored in non-transient computer memory, comprising a module identifying targeted drug treatments for treatment of a phenotype in a subject, the module comprising:
a first set of instructions automatically determining biomolecules affected by the phenotype by isolating differentially expressed biomolecules associated with each of the phenotypes;
a second set of instructions automatically determining a first probability value that is a probability of observing the number of differentially expressed biomolecules in a biological pathway just by chance;
a third set of instructions automatically determining a second probability value associated with a total accumulation of the differentially expressed genes as measured in the biological pathway;
a fourth set of instructions automatically combining the first probability value and the second probability value so as to calculate a unique pathway-specific uncorrected value for the biological pathway;
a fifth set of instructions identifying upstream;
a sixth set of instructions, using a putative mechanism inference, to determine mechanisms involved with the plurality of differentially expressed biomolecules;
a seventh set of instructions determining at least one targeted drug treatment configured to reverse expressions of at least two biomolecules found to be differentially expressed in the phenotype; and
an eighth set of instructions displaying the targeted drug treatment determination on an output.
